(19) United States
(12) Patent Application Publication
(10) Pub. No.: US 2020/0046209 A1
(43) Pub. Date: Feb. 13, 2020

(54) ENDOSCOPE SHAFT

(71) Applicant: AdaptivEndo LLC, Louisville, KY (US)

(72) Inventor: Hershel E. Fancher, Georgetown, IN (US)

(21) Appl. No.: 16/599,230

(22) Filed: Oct. 11, 2019

Related U.S. Application Data
(63) Continuation of application No. PCT/US18/25786, filed on Apr. 3, 2018.
(60) Provisional application No. 62/485,433, filed on Apr. 14, 2017.

(51) Int. Cl. A61B 1/018; A61B 1/00; A61B 1/005; A61B 1/05; A61B 1/06

(52) U.S. Cl. CPC A61B 1/018; A61B 1/00103; A61B 1/0057; A61B 1/00137; A61B 1/06; A61B 1/0055; A61B 1/0011; A61B 1/00119; A61B 1/05

(57) ABSTRACT

An endoscope is provided. The endoscope incorporates a disposable flexible endoscope shaft that comprises an insertion tube and a distal articulating section. The insertion tube comprises a central core, two or more pull wires, each located with a concentric compression coil, an outer braid, and an outer sheath. The distal articulating section comprises a deformable element for articulating the distal tip. In an alternative embodiment, the distal articulating section comprises of a plurality of articulating links that, when assembled, create a plurality of concentric tab and socket pivot joints, said plurality of concentric tab and socket joints being alternatingly located in two perpendicular planes when the central axis of all links are aligned. Each concentric tab and socket joint providing the means for rotation in a single axis. Methods of manufacturing are also disclosed.

### FIELD OF THE INVENTION

This invention generally relates to endoscopes and more particularly to endoscopes used in gastroenterological applications.

### BACKGROUND OF THE INVENTION

Endoscopes are used in a wide variety of medical procedures to visualize internal cavities or potential spaces within the human body during diagnostic or therapeutic procedures.

One factor affecting endoscope design and use is infectious disease transmission. Although infectious disease transmission has always been a risk with reuseable endoscopes, this risk has increased with the advent of antibiotic resistant bacteria. Once transmission of these bacteria has occurred between patients, antibiotic resistance makes it difficult to treat these infections. Many of these bacteria, including staphylococcus, have the ability to form a protective outer barrier, frequently referred to as a bio-film, which protects the infectious bacteria from during cleaning procedures.

Another recent development that increases the risk of infectious disease transmission is an ever-increasing number of diagnostic procedures that require the use of smaller more complicated instruments and longer working lumens and distal manipulators, e.g. elevators, associated with the endoscope distal articulating shaft. Together, the increasing prevalence of difficult to treat infectious diseases and more complex difficult to clean endoscopes make the sterilization and re-use of endoscopes an increasingly risky activity.

Reducing or limiting the potential for infectious disease transmission is desirable. Accordingly, new endoscope designs are desired.

### SUMMARY

Disposable flexible endoscope shafts of the present disclosure comprise a proximal portion having an insertion tube assembly and a distal portion having an articulating section assembly.

Insertion tube assemblies disclosed herein include a central core with lumens for the instrument tube, air/water tube, camera wire harness, and/or LED wire harness. One or more of the lumens can be positioned entirely within the periphery of the central core and have a closed circumference. Additionally or alternatively, one or more of the lumens can have a circumferential opening that lies along the periphery of the central core.

The central core has an outer surface. The outer surface can define a plurality of compression coil grooves arranged to receive compression coils. In a particular example, an outer surface of the central core defines four compression coil grooves.

The plurality of compression coil grooves of the central core can be equally spaced around the periphery (e.g., circumference) of the central core. For example, four compression coil grooves can be spaced 90° apart around the periphery of the central core. One or more of the compression coil grooves can be positioned along a radial direction that extends between lumens for the instrument tube, air/water tube, camera wire harness, and/or LED wire harness.

The compression coil grooves are arranged to receive compression coils and/or articulation wires. The compression coils are preferably arranged to increase the compressive strength of the central core along a longitudinal direction. The compression coils can be helically wound metal wires and/or spiral cut cannulas that provide for lateral flexibility. The articulation wires can be slidably positioned within lumens defined by the compression coils.

The insertion tube assembly can include a sleeve (e.g., braided sleeve) positioned around the central core. The sleeve can be arranged to retain the compressions coils and/or articulation wires in the compression coil grooves of the central core. Additionally or alternatively, the sleeve can be arranged to provide torsional strength to the insertion tube assembly. The braided sleeve can include a metal braid or plastic braids such as PET.

Insertion tube assembly comprises an outer sheath positioned around sleeve. The outer sheath may be applied as a reflowed tube or by an extruder. The outer sheath can bond to the sleeve and/or to the central core through apertures of the sleeve.

The articulating section assembly comprises an articulating section having a plurality of hinges. Each hinge provides rotation around a pivot axis. The pivot axes of hinges can extend transverse to a longitudinal axis of the articulating section. Additionally, the pivot axis of one or more of the hinges can be in a different plane than the pivot axis of one or more other hinges. For example, the pivot axes of hinges can be in alternatively located in planes perpendicular to one another when the articulating section is in a straight (e.g., unbent) configuration. Advantageously, such an arrangement can provide an articulating element capable of articulating the distal tip/camera in three dimensions.

One or more hinges of the articulating section can be living hinges. In some instances, the articulating section is a unitary articulating section form. The unitary articulating section can be formed from a single piece of material. The unitary articulating section structure can be fabricated using injection molding or additive material fabrication techniques. Alternatively, the unitary articulating section can be formed by extruding a cylinder and cutting the cylinder tube with a knife, laser, milling tool, water jet, or other material removal mechanism to form the living hinges. As will be appreciated, the bending and torque fidelity characteristics of the articulating section can be configured by configuring the angles of the cuts/recesses that define the hinges and/or the distance between adjacent hinges.

In another arrangement, the articulating section structure comprises a plurality of discrete links that, when assembled, define a plurality of concentric tab and socket pivot joints that function as a hinge. As mentioned above, each hinge (e.g., tab and socket pivot join) can provide for rotation around a pivot axis in a single plane. Moreover, the plurality of concentric tab and socket joints can be alternatingly located in two perpendicular planes when the central axis of all links are aligned so as to provide the articulating section with multiple degrees of freedom.

An outer sheath can be positioned around the articulating section to prevent contaminants from entering the one or more hinges and/or lumens defined by the articulating section. The articulating section can include a distal cap defining an air/water nozzle, an instrument tube outlet, a camera outlet, and/or an LED outlet.

The insertion tube assembly and articulating section can be bonded together (e.g., heat or friction welding, adhesive, etc) and/or attached together with mating features on the contacting surface (e.g., threads) or with a transition tube, as shown in the illustrated embodiment. The mid-plane of the transition tube can be located at the transition between the insertion tube assembly and the articulating section, and the transition tube can be bonded (e.g., swaged or adhered with adhesive) onto both the insertion tube assembly and the distal articulating section to form a secure attachment. The transition tube can be deformable to allow deflection of the flexible endoscope shaft at the transition.

The insertion tube assemblies disclosed herein can be manufactured using a continuous reel-to-reel manufacturing process. Compression coils and/or articulation wires can be inserted into each of compression coil grooves of the central core using a continuous reel-to-reel process. The braided sleeve can be applied around the central core, compression coils, and/or articulation wires during the continuous reel-to-reel manufacturing process. Additionally, or alternatively, the outer sheath can be applied during the continuous reel-to-reel manufacturing process to an assembly of the central core, compression coils and/or articulation wires, and braided sleeve. For example, the assembly can be passed through an extrusion mold during the continuous reel-to-reel manufacturing process so as to apply the outer sheath to the portion of the insertion tube assembly. Such a process can create a smooth outer sheath that is integrally bonded to the central core, braided sleeve, and compression coils.

Advantageously, providing reel-to-reel manufacturing of an insertion tube assembly can reduce the cost of manufacturing the endoscope shaft assembly and increase production speed. Accordingly, in certain aspects, the present disclosure provides a low-cost, flexible endoscope shaft and method of manufacturing same. As the insertion tube assembly can be manufactured continuously, desired lengths of insertion tube assembly, or a portion thereof, can be cut to length at the end of the outer sheath extrusion process or cut from a finish good reel. These reel-to-reel techniques for fabricating the insertion tube avoids braiding and coating the insertion tube in discrete sections using labor intensive processes.

As will be appreciated, the insertion tube assemblies disclosed can provide containment of wiring, tubes, and actuation wires of the endoscope shaft while having torsional and compressive strength sufficient to advance the articulating section assembly through tortuous vessels of a patient.

The inventive aspects and embodiments discussed below in the following separate paragraphs of the summary may be used independently or in combination with each other.

Further forms, objects, features, aspects, benefits, advantages, and embodiments of the present invention will become apparent from a detailed description and drawings provided herewith.

### BRIEF DESCRIPTION OF THE DRAWINGS

FIG. 1 illustrates a perspective view of a disposable flexible endoscope shaft assembly.

FIG. 2A illustrates a perspective view of an insertion tube assembly.

FIG. 2B illustrates a perspective view of a distal end of the insertion tube assembly.

FIG. 3 illustrates an exploded view of the insertion tube assembly.

FIG. 4A illustrates a perspective view of a central core.

FIG. 4B illustrates a perspective view of the distal end of the central core.

FIG. 5 illustrates a perspective view of the articulating section assembly.

FIG. 6A illustrates an exploded view of the articulating section assembly.

FIG. 6B is a partial exploded view of the articulating section assembly.

FIG. 7A illustrates a perspective view of the unitary articulating section in a straight configuration.

FIG. 7B illustrates a perspective view of the unitary articulating section in a deflected configuration.

FIG. 7C illustrates a close-up view of the unitary articulating section.

FIG. 8A illustrates a close-up of the proximal end of the unitary articulating section of FIGS. 7A-7C.

FIG. 8B illustrates a close-up of the distal end of the unitary articulating section of FIGS. 7A-8A.

FIG. 9 illustrates a perspective view of an articulating link assembly.

FIG. 10A illustrates a perspective view of an articulating link.

FIG. 10B illustrates a perspective view of the proximal articulating link.

FIG. 10C illustrates a perspective view of the distal articulating link.

FIG. 11 illustrates a perspective view of the articulation pull wire and termination ring assembly.

FIG. 12 is a flowchart illustrating a process for manufacturing disposable flexible endoscope shafts disclosed herein.

FIG. 13 illustrates a first portion of a manufacturing arrangement.

FIG. 14 illustrates a second portion of a manufacturing arrangement.

### DETAILED DESCRIPTION OF THE INVENTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

As used herein, “proximal” refers to an end or direction associated with a physician or other treating personnel during a device operation, and “distal” refers to the opposite end (“patient end/treating end”). The drawing figures referred to herein are provided for illustrative purposes only. They should not be construed as limiting the scope of the invention defined by the claims, including that they may not necessarily be drawn to scale.

FIG. 1 illustrates a disposable flexible endoscope shaft assembly 10 having a distal portion comprising a articulating section assembly 20 and a proximal portion comprising an insertion tube assembly 30.

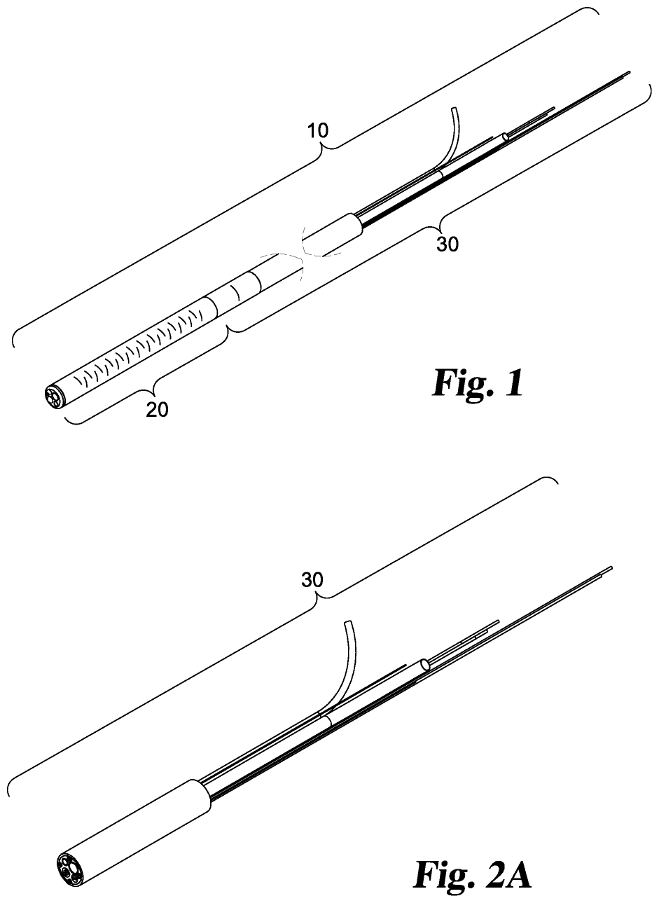

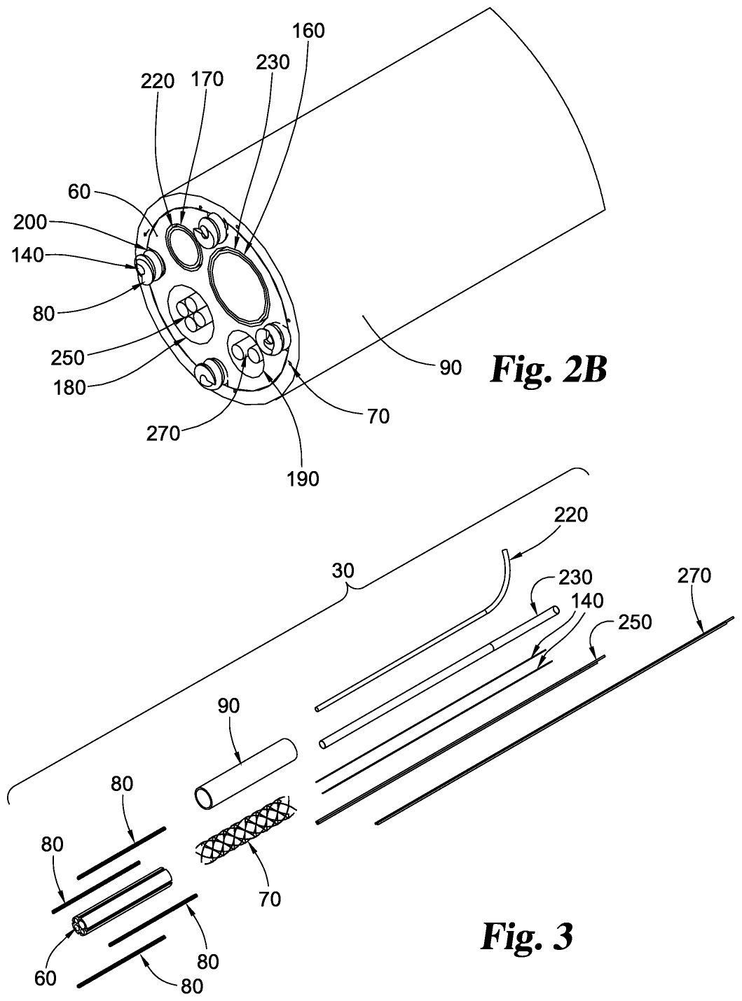

Turning now to FIGS. 2A, 2B and 3, the insertion tube assembly 30 includes a central core 60. The central core 60 includes an instrument tube lumen 160 that receives an instrument tube 230, an air/water tube lumen 170 that receives an air/water tube 220, a camera lumen 180 that receives a camera wire harness 250, and a light emitter lumen 190 that receives an light emitter wire harness 270.

An articulation wire 140 and a compression coil 80 are located in a compression coil groove 200 defined by central core 60. As will be discussed with more detail below, these can be positioned at locations spaced around the perimeter of the central core 60.

A braided sleeve 70 surrounds the central core 60 and is positioned between the central core and an outer sheath 90. The outer sheath 90 can be applied over the braided sleeve 70 an central core 60 as a reflowed tube (e.g., thermal lamination) or by extrusion.

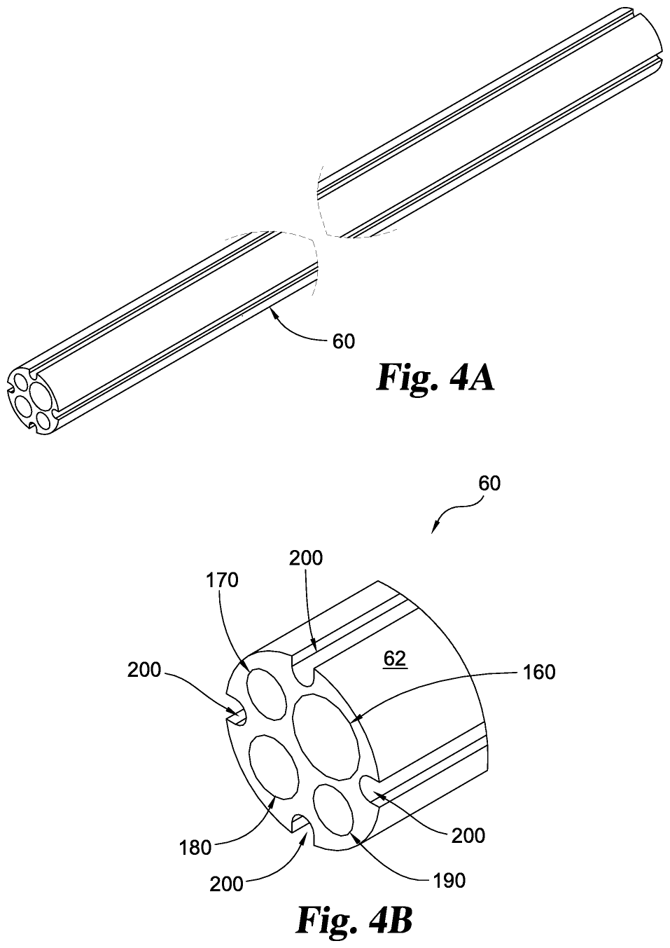

FIGS. 4A and 4B depict the central core 60. Central core 60 has an outer surface 62 defining compression coil grooves 200 arranged to receive compression coils. In the illustrated embodiment, outer surface 62 of central core 60 defines four compression coil grooves 200 spaced evenly around the periphery of central core 60. As mentioned previously, central core 60 defines an instrument tube lumen 160, an air/water tube lumen 170, a camera lumen 180, and an light emitter lumen 190.

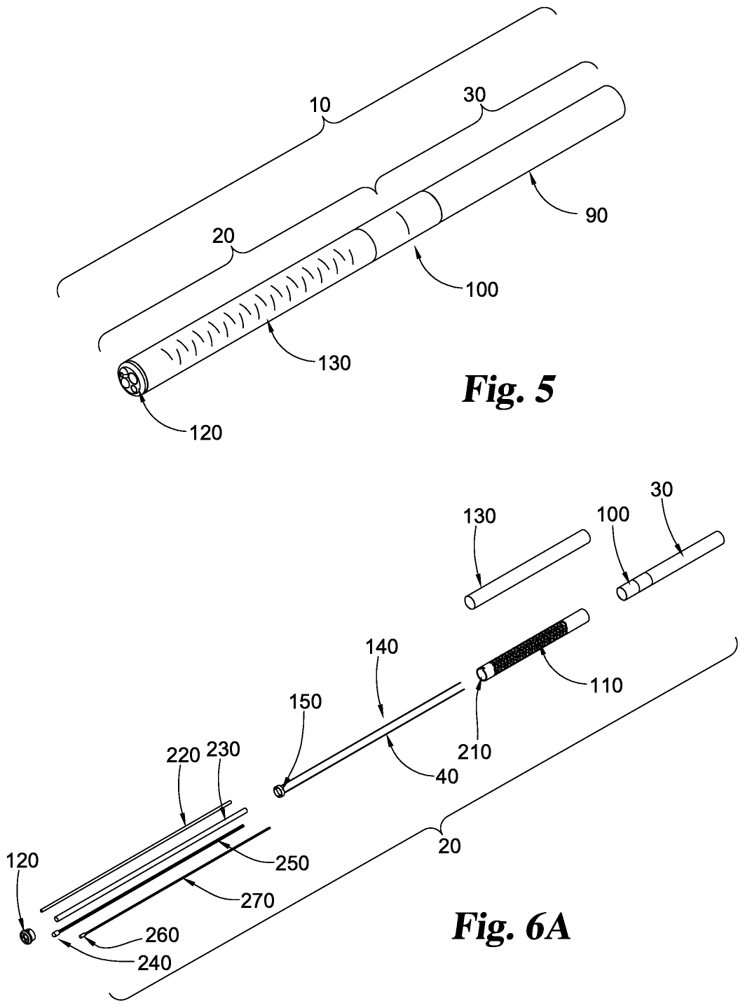

FIG. 5 illustrates a cap 120 at the distal end of the articulating section 20 and an articulating section outer sheath 130. The articulating section assembly 20 is connected to the insertion tube assembly 30 with a transition tube 100. The transition tube 100 may be mechanically secured to both the articulating section assembly 20 and the insertion tube assembly 30 through a process such as swaging.

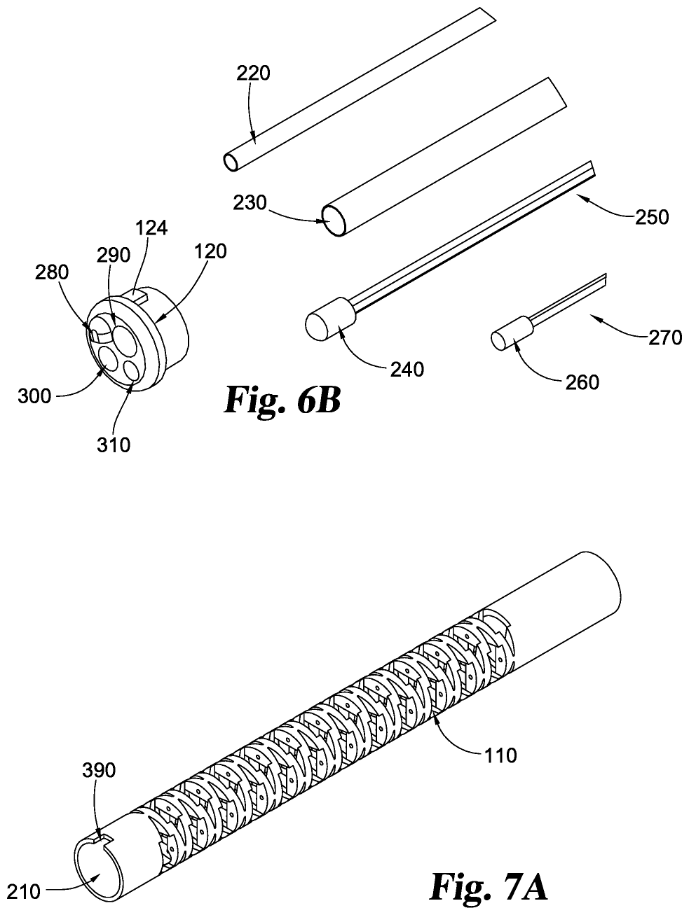

FIGS. 6A and 6B depict the articulating section assembly 20. The articulating section assembly 20 comprises an articulation wire and termination ring assembly 40 having a termination ring 150 positioned at a distal end of articulation wires 140, a unitary articulating section 110, an articulating section outer sheath 130, cap 120, an instrument tube 230, an air/water tube 220, a camera 240 and camera wiring harness 250, a light emitter 260 (e.g., an LED) and light emitter wiring harness 270. When disposable flexible endoscope shaft assembly 10 is assembled, air/water tube 220 extends through a lumen 210 defined by the articulating section 110 and through air/water tube lumen 170, instrument tube 230 extends through lumen 210 and through instrument tube lumen 160, camera wiring harness 242 extends through lumen 210 and through camera lumen 180, and light emitter wire harness 270 extends through lumen 210 and through light emitter lumen 190.

Cap 120 defines an air/water nozzle 280, an instrument tube outlet 290, a camera outlet 300, and a light emitter outlet 310. Cap 120 includes a cap alignment tab 124 arranged to engage a cap alignment notch of an articulating section (e.g., unitary articulating section 110).

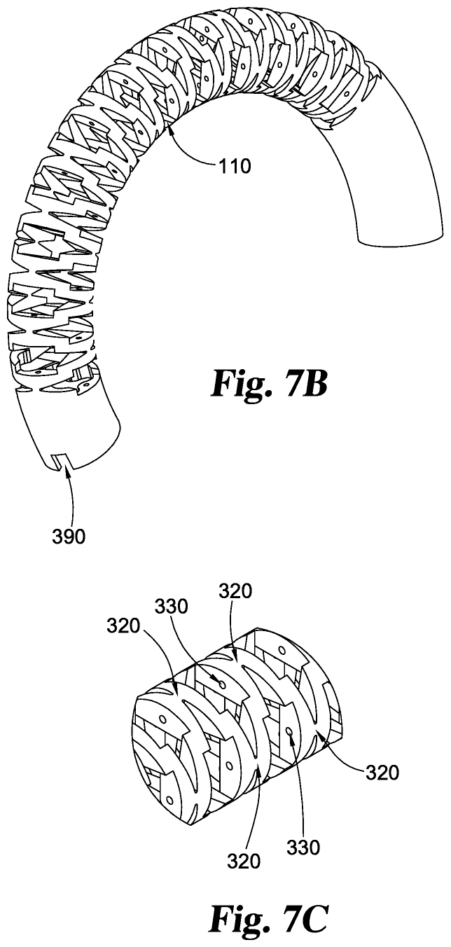

FIGS. 7A and 7B depict the unitary articulating section 110 comprising a plurality of living hinges 320 being alternatingly located in two perpendicular planes when unitary articulating section is in a straight configuration. Each deformable living hinge element 320 provides means for rotation around a pivot axis in a single plane. An articulation wire lumen 330 traverses each living hinge element 320 and is arranged to receive an articulation wire 140.

The lumen 210 located within the unitary articulating section 110 can receive an air/water tube, an instrument tube, and/or wiring 210. A cap alignment notch 390 is located at the distal end of the unitary articulating section 110 and arranged to receive the cap alignment tab 124.

FIG. 7B illustrates the unitary articulating section 110 in a deformed configuration consistent with an articulation of 180° for retrograde viewing using the distally mounted camera 240.

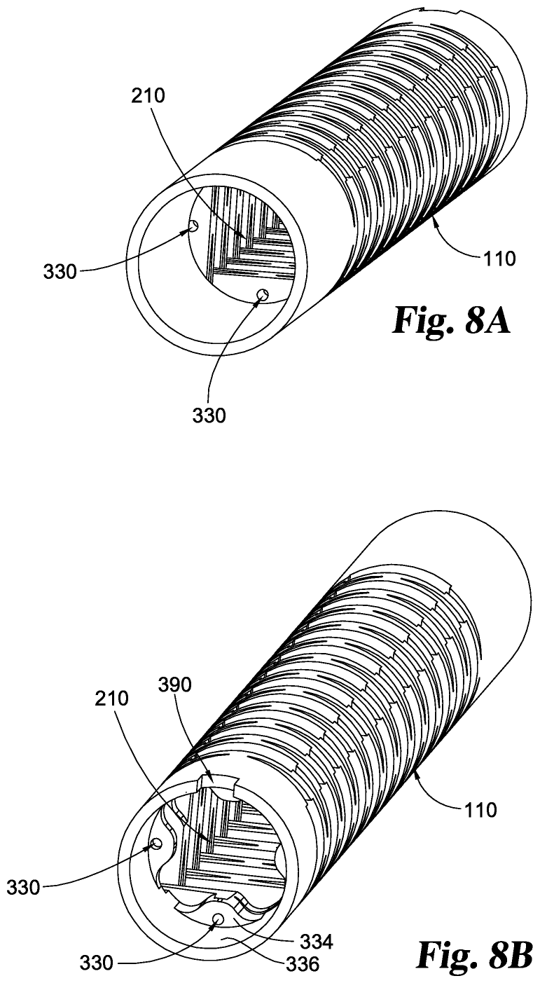

FIG. 8A illustrates the proximal end of the unitary articulating section 110, and FIG. 8B illustrates the distal end of the unitary articulating section 110. As seen in FIG. 8B, a distally facing surface 334 extends inwardly from the inner surface 336 of the unitary articulating section 110. Distally facing surface 334 is arranged to contact termination ring 150 and transfer tensile force from the articulation wire and termination ring assembly 40 to the unitary articulating section 110.

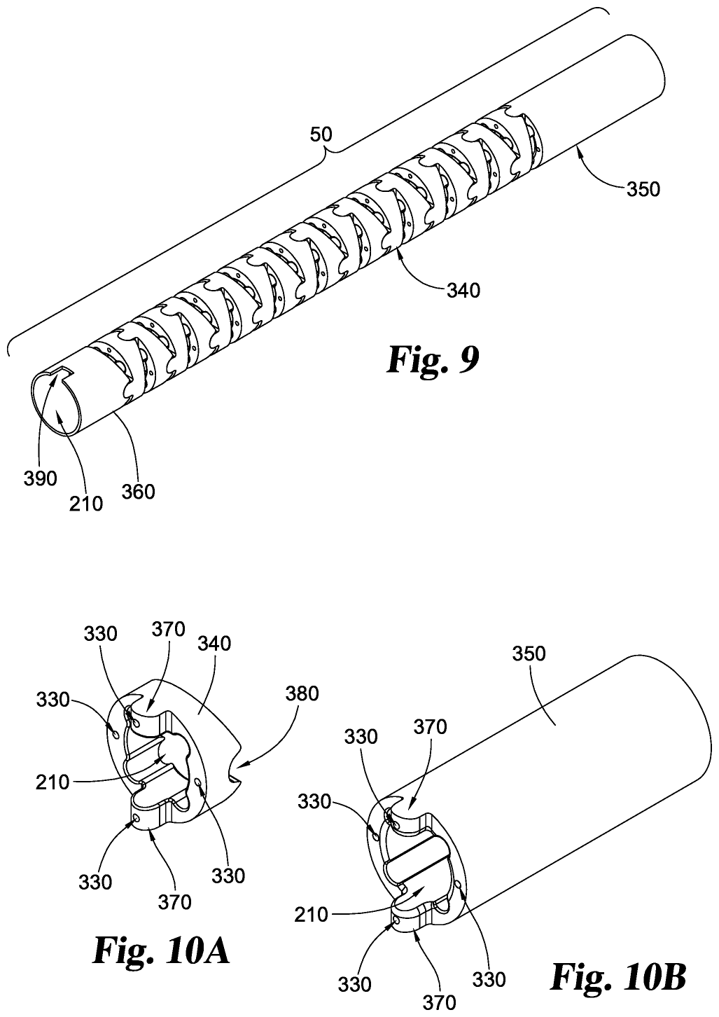

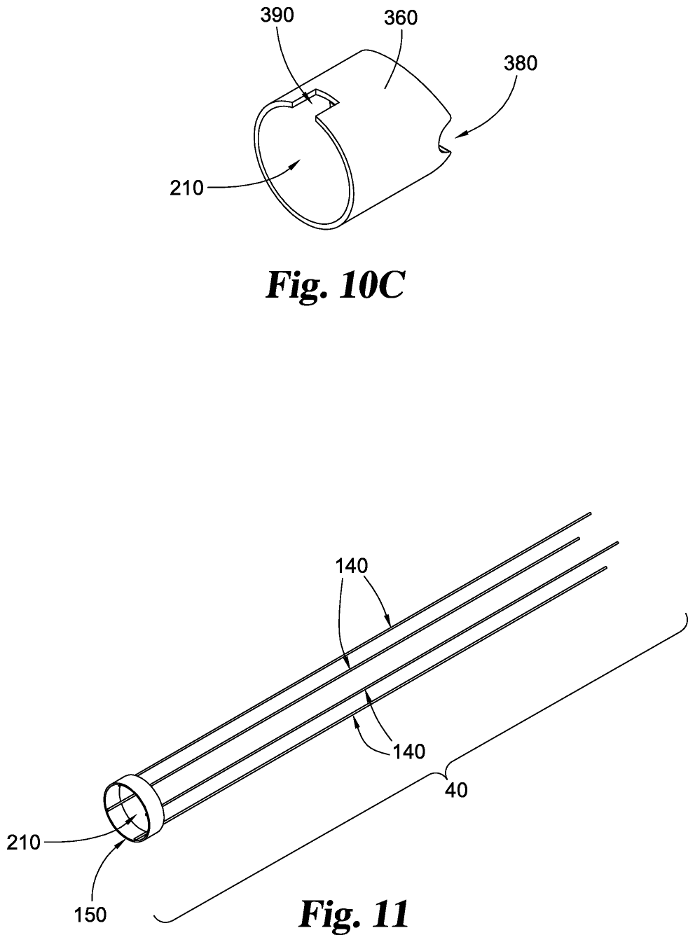

FIGS. 9-10C illustrate an articulating link assembly 50 comprising a proximal articulating link 350, an intermediate articulating link 340, and a distal articulating link 360. The proximal articulating link 350, intermediate articulating link 340, and distal articulating link 360 each define a lumen 210 arranged to receive an air/water tube, an instrument tube, and/or wiring.

The proximal articulating link 350 includes pivot tabs 370 located in a first (e.g., vertical) plane. The intermediate articulating link 340 comprises articulation pull wire lumens 330, pivot tabs 370 located in a first plane, and pivot sockets 380 located in a second plane. The distal articulating link 360 includes a cap alignment notch 390 to control alignment of the camera 240 relative to each of the four articulation pull wires 140 and two pivot sockets 380 located in the first or second plane. When assembled, the pivot tabs 370 are received within and pivotable relative to the pivot sockets 380.

FIG. 11 illustrates the articulation pull wire and termination ring assembly 40 comprising four articulation pull wires 140 and an articulation pull wire termination ring 150. The interior of the articulation pull wire termination ring 150 defines lumen 210 for passage of air/water tube, instrument tube, and wiring.

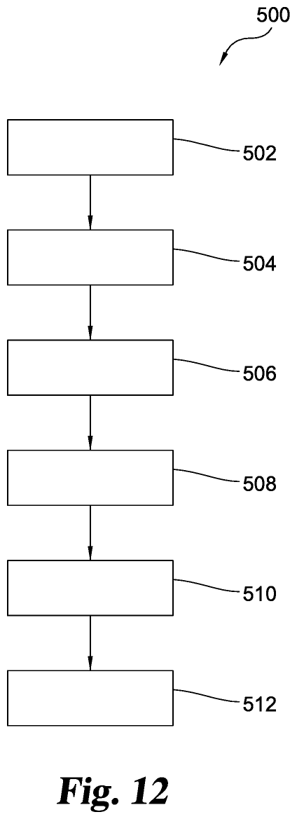

Turning now to FIG. 12, a process 500 for manufacturing insertion tube assemblies disclosed herein is described. The insertion tube assemblies can be manufactured using a continuous reel-to-reel manufacturing process. The process can being in stage 502 wherein reels of compression coils and/or articulation wires are provided. In stage 504, the compression coils and/or articulation wires are inserted into each of compression coil grooves of the central core. As the compression coil grooves are on the outer surface of the central core, the compression coils and/or articulation wires can be joined with the central core from a lateral direction in a continuous process. In some instances, the compression coils and/or articulation wires are advanced through a mold and the central core is extruded around the compression coils and/or articulation wires.

In stage 506, the braided sleeve can be applied around the central core, compression coils, and/or articulation wires. Again, this can occur during a continuous reel-to-reel manufacturing process with the central core, compression coils, and/or articulation wires continually being assembled and then advanced to receive the braided sleeve.

In stage 508, the outer sheath can be applied. This, again, can occur during a continuous reel-to-reel manufacturing process. For example, the central core, compression coils, and/or articulation wires, and braided sleeve assembly can pass through an extrusion mold that extrudes the outer sheath around the assembly. Such a process can create a smooth outer sheath that is integrally bonded to the central core, braided sleeve, and compression coils.

In stage 510, the desired length of insertion tube assembly, or a portion thereof, can be cut to length to length. This can occur immediately after the outer sheath extrusion process or, alternatively, from a reel of finished goods. The process concludes in stage 512. As will be appreciated by those skilled in the art, this reel-to-reel technique for fabricating the insertion tube avoids labor and time intensive batch processes currently used to form discrete sections of endoscope shafts. Applicant believes this can reduce the costs of manufacturing endoscope shaft assemblies and/or increase production speed.

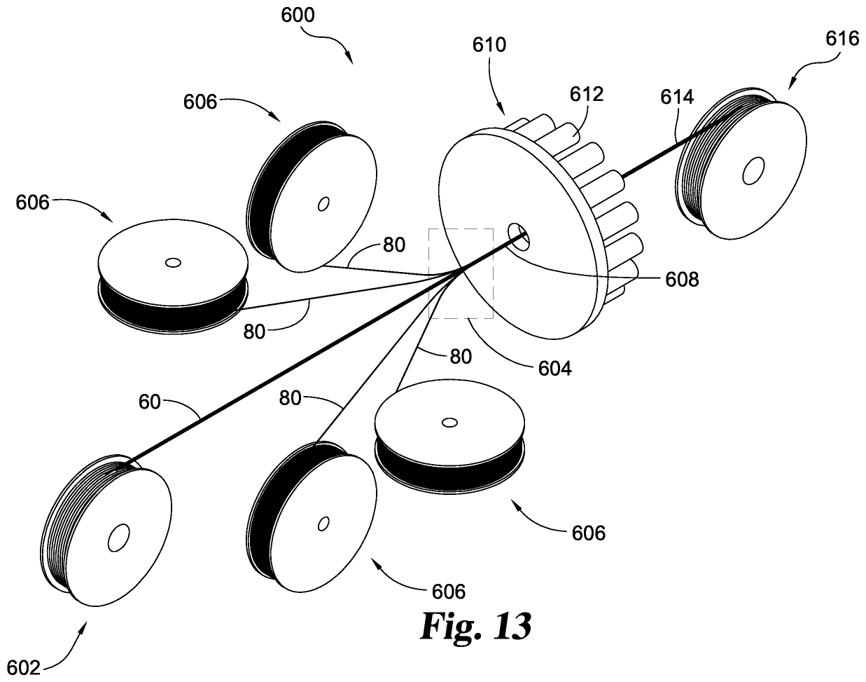

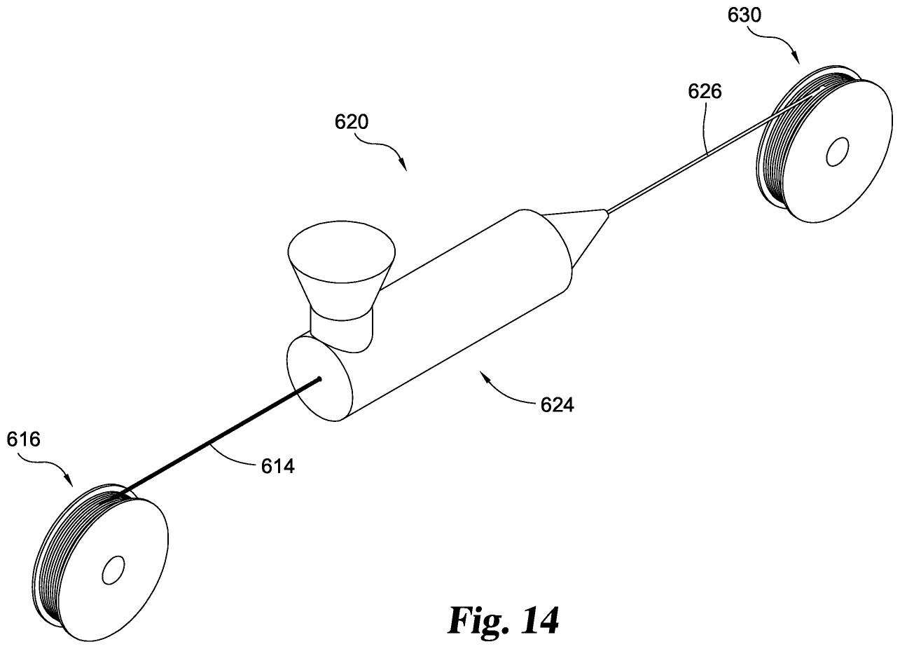

FIGS. 13 and 14 illustrate a manufacturing arrangement suitable for practicing the process described above. In FIG. 13, a first portion 600 of the manufacturing arrangement is shown. First portion 600 includes a central core 60 extending from a central core reel 602 to a first fixture 604 and compression coils 80 extend from compression coil reels 606 into first fixture 604. First fixture 604 is arranged to press the compression coils 80 into compression coil grooves 20 of the central core 60, as the compression coils 80 and central core 60 are continuously unwound from the reels and advanced through first fixture, to form a central core and compression coil assembly 608.

From first fixture 604, the central core and compression coil assembly 608 passes through a second fixture 610. Second fixture 610 is arranged to form a braided sleeve around the central core and compression coil assembly as the central core and compression coil assembly continually advances through second fixture 610. For example, second fixture 610 may include a braiding machine having a plurality of bobbins 612 that weave relative to one another to form a braided (e.g., woven) sleeve around the central core and compression coil assembly. The central core, compression coil, and braided sleeve assembly 614 can then extend to a reel 616.

FIG. 14 illustrates a second portion 620 of the manufacturing arrangement. In second portion 620, the central core, compression coil, and braided sleeve assembly 614 extends from reel 616 to extrusion mold 624. Extrusion mold 624 is arranged to continuously extrude an outer sheath around the assembly 614 as the assembly is advanced therethrough. The outer sheath assembly 626 extends from the extrusion mold 624 to a finished goods reel 630.

While one manufacturing arrangement has been illustrated and described, the present disclosure is not limited to such. For example, first fixture 604 and second fixture 610 can be combined into a single fixture. Similarly, reel 616 may be omitted and the central core, compression coil, and braided sleeve assembly 614 extend directly from the second fixture 610 or combined single fixture to the extrusion mold 624.

The following numbered clauses set out specific embodiments that may be useful in understanding the present invention:

1. A disposable flexible endoscope shaft, comprising: a proximal insertion tube and a distal articulating section, wherein proximal insertion tube is comprised of an inner extrusion, two or more pull wires, each located with a concentric compression coil, an outer braid, and an outer smooth sheath.

2. The disposable flexible endoscope shaft of clause 1, wherein the distal section of the shaft is comprised of a single deformable element that provides the structure for articulating the distal cap that incorporates a camera.

3. The disposable flexible endoscope shaft of clause 1, wherein the distal section of the shaft is comprised of a plurality of articulating links that incorporate concentric tab and socket pivot joints and provides the structure for articulating the distal cap that incorporates a camera.

4. The disposable flexible endoscope shaft of clause 3, wherein the most proximal and most distal articulating links are unique from the plurality of central articulating links and possess unique features for connecting to the proximal insertion shaft and the distal cap, respectively.

5. A method of forming an endoscope shaft, comprising:

inserting a compression coil into a compression coil groove of a central core to form a compression coil and central core assembly; and

braiding a braided sleeve around the compression coil and central core assembly to form a braided assembly;

wherein the inserting and braiding are performed during a continuous reel-to-reel process.

6. The method of clause 5 further comprising:

molding an exterior sheath around the braided assembly.

7. The method of clause 6, wherein the molding comprises advancing the braided assembly through an opening in a die of an extrusion mold so as to continuously apply an outer sheath to the braided assembly in a reel-to-reel process.

8. An endoscope shaft comprising:

a central core defining an instrument lumen, an air/water lumen, a light emitter lumen, and a camera lumen;

said central core having an outer surface defining a first compression coil groove arranged for receiving a first compression coil and a first articulation wire, a second compression coil groove arranged for receiving a second compression coil and a second articulation wire, a third compression coil groove arranged for receiving a third compression coil and a third articulation wire, and a fourth compression coil groove arranged for receiving a fourth compression coil and a fourth articulation wire;

wherein said first compression coil groove, said second compression coil groove, said third compression coil groove, and said fourth compression coil groove are spaced around an outer periphery of the central core.

9. The endoscope shaft of clause 8 wherein:

said first compression coil groove, said second compression coil groove, said third compression coil groove, and said fourth compression coil groove are spaced equally apart around the outer periphery of the central core.

10. The endoscope shaft of clause 8 or 9 further comprising:

a first compression coil and a first articulation wire received within the first compression coil groove, a second compression coil and a second articulation wire received within the second compression coil groove, a third compression coil and a third articulation wire received within the third compression coil groove, and a fourth compression coil and a fourth articulation wire received within the fourth compression coil groove to form a central core assembly.

11. The endoscope shaft of clause 10 further comprising:

a braided sleeve positioned around the central core assembly to form a braided assembly.

12. The endoscope of clause 11 further comprising:

an outer sheath positioned around the braided assembly.

13. The endoscope shaft of any one of clauses 8-12 further comprising:

an instrument tube received within the instrument tube lumen;

an air/water tube received within the air/water tube lumen;

a camera wire harness received within the camera lumen; and

a light emitter wire harness received within the light emitter lumen.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A disposable flexible endoscope shaft, comprising: a proximal insertion tube and a distal articulating section, wherein proximal insertion tube is comprised of an inner extrusion, two or more pull wires, each located with a concentric compression coil, an outer braid, and an outer smooth sheath.

2. The disposable flexible endoscope shaft of claim 1, wherein the distal section of the shaft is comprised of a single deformable element that provides the structure for articulating the distal cap that incorporates a camera.

3. The disposable flexible endoscope shaft of claim 1, wherein the distal section of the shaft is comprised of a plurality of articulating links that incorporate concentric tab and socket pivot joints and provides the structure for articulating the distal cap that incorporates a camera.

4. The disposable flexible endoscope shaft of claim 3, wherein the most proximal and most distal articulating links are unique from the plurality of central articulating links and possess unique features for connecting to the proximal insertion shaft and the distal cap, respectively.

5. A method of forming an endoscope shaft, comprising:
inserting a compression coil into a compression coil groove of a central core to form a compression coil and central core assembly; and
braiding a braided sleeve around the compression coil and central core assembly to form a braided assembly;
wherein the inserting and braiding are performed during a continuous reel-to-reel process.

6. The method of claim 5 further comprising:
molding an exterior sheath around the braided assembly.

7. The method of claim 6, wherein:
the molding comprises advancing the braided assembly through an opening in a die of an extrusion mold so as to continuously apply an outer sheath to the braided assembly in a reel-to-reel process.

8. An endoscope shaft comprising:
a central core defining an instrument lumen, an air/water lumen, a light emitter lumen, and a camera lumen;
said central core having an outer surface defining a first compression coil groove arranged for receiving a first compression coil and a first articulation wire, a second compression coil groove arranged for receiving a second compression coil and a second articulation wire, a third compression coil groove arranged for receiving a third compression coil and a third articulation wire, and a fourth compression coil groove arranged for receiving a fourth compression coil and a fourth articulation wire;
wherein said first compression coil groove, said second compression coil groove, said third compression coil groove, and said fourth compression coil groove are spaced around an outer periphery of the central core.

9. The endoscope shaft of claim 8 wherein:
said first compression coil groove, said second compression coil groove, said third compression coil groove, and said fourth compression coil groove are spaced equally apart around the outer periphery of the central core.

10. The endoscope shaft of claim 8 further comprising:
a first compression coil and a first articulation wire received within the first compression coil groove, a second compression coil and a second articulation wire received within the second compression coil groove, a third compression coil and a third articulation wire received within the third compression coil groove, and a fourth compression coil and a fourth articulation wire received within the fourth compression coil groove to form a central core assembly.

11. The endoscope shaft of claim 10 further comprising:
a braided sleeve positioned around the central core assembly to form a braided assembly.

12. The endoscope of claim 11 further comprising:
an outer sheath positioned around the braided assembly.

13. The endoscope shaft of claim 8 further comprising:
an instrument tube received within the instrument tube lumen;
an air/water tube received within the air/water tube lumen;
a camera wire harness received within the camera lumen; and
a light emitter wire harness received within the light emitter lumen.
